(19) United States
(12) Patent Application Publication
(10) Pub. No.: US 2019/0010213 A1
(43) Pub. Date: Jan. 10, 2019

(54) COLLAGEN-BASED BIOMATERIAL

(71) Applicant: Research Foundation of the City University of New York, New York, NY (US)

(72) Inventors: Yujia Xu, New York, NY (US); Parminder Jeet Kaur, New York, NY (US); FangFang Chen, New York, NY (US)

(21) Appl. No.: 16/030,197

(22) Filed: Jul. 9, 2018

Related U.S. Application Data
(60) Provisional application No. 62/529,761, filed on Jul. 7, 2017.

(51) Int. Cl. C07K 14/78

(52) U.S. Cl. CPC C07K 14/78

(57) ABSTRACT

A fibril-forming peptide having a structure of f_{N}-[An₁]-(Gly-X-Y)ₙ-[Ac₁]-L-[An₂]-(Gly-X-Y)ₙ-[Ac₂]-L-f_{C} where L is (Gly-Pro-Z)ⱼ and Z is Pro or Hyp. [An₁], [An₂], [Ac₁] and [Ac₂] are each chains of 0-3 amino acid residues. The peptide self-assembles to form a collagen-like material.

### CROSS-REFERENCE TO RELATED APPLICATIONS

This application claims priority to and is a non-provisional of U.S. Patent Application 62/529,761 (filed Jul. 7, 2017), the entirety of which is incorporated herein by reference.

### STATEMENT OF FEDERALLY SPONSORED RESEARCH OR DEVELOPMENT

This invention was made with government support under grant number CHE-1022120 awarded by the National Science Foundation. The government has certain rights in the invention.

### REFERENCE TO A SEQUENCE LISTING

This application refers to a “Sequence Listing” listed below, which is provided as an electronic document submitted herewith. This electronic document is incorporated herein by reference in its entirety.

### BACKGROUND OF THE INVENTION

Collagen-based materials have served humanity for nearly 200 years. Among many unique physical and molecular properties, collagen is especially prized for its biocompatibility and for its tensile strength. Both properties are attributes of collagen fibrils—the functional form of collagens in tissues and organs. Collagen fibrils are the major component of the molecular scaffold of the extracellular matrix, and maintain the microenvironment for cells during tissue growth and function. Collagen-based biomaterials are projected to be a 41-billion-dollar industry by 2020, with a compound annual growth rate (CAGR) of 16% based on the recent BENZINGA® business analysis. Collagen-based materials have been used extensively in medicine, pharmaceuticals, personal care cosmetics, food industry and leather industry.

Traditional collagen-based materials rely on collagens extracted from animal tissues, and frequently from the byproducts of meat industry. The purification and extraction process are often costly and utilize harsh or even toxic chemicals. The incidences of transmission of bovine spongiform encephalopathy (BSE) have also raised serious health concerns of using collagens from animals for medical use or for personal care products. With the development of the recombinant DNA technology, a new industry emerges to produce collagens from expression systems such as yeast, tobacco or mammalian cell lines. These collagens are generally safe from cross-contamination of pathogens from host animals, and are more environment-friendly. The expression productions so far have been constrained to reproduce the full-chain collagens, which is a biologically costly process and often suffers from the low yield. These materials also have the disadvantage of being difficult to tailor for specific tissue applications.

The discussion above is merely provided for general background information and is not intended to be used as an aid in determining the scope of the claimed subject matter.

### BRIEF DESCRIPTION OF THE INVENTION

A fibril-forming peptide having a structure of f_{N}-[An₁]-(Gly-X-Y)ₙ-[Ac₁]-L-[An₂]-(Gly-X-Y)ₙ-[Ac₂]-L-f_{C} is provided, where Lᵢ is (Gly-Pro-Z)ⱼ where Z is Pro or Hyp, [An₁], [An₂], [Ac₁] and [Ac₂] are each chains of 0-3 amino acid residues in any sequence. The peptide self-assembles to form collagen-like fibrils. An advantage that may be realized in the practice of some disclosed embodiments of the peptide is that the peptide need not be isolated from animal products, and both the size and the amino acid sequences can be custom designed for specific applications.

In a first embodiment, a fibril forming peptide is provided. The peptide has a primary structure given by:

f_{N}-([An]-[Ag]-[Ac]-Lᵢ)ᵢ-f_{C}

wherein the [Ag] is an amino acid sequence having between 6 and 200 residues wherein every third residue in the [Ag] is Gly; wherein i gives a number of repeating units such that i=2 or i>3; f_{N} is an N-terminal overhang region having between 9 and 50 amino acids; f_{C} is an C-terminal overhang region having between 9 and 50 amino acids; L is a linker given by (Gly-Pro-Z)ⱼ where j is an integer such that 2≤j, Z is Pro or Hyp; and [An] is a chain of 0-3 amino acid residues, [Ac] is a chain of 0-3 amino acid residues; wherein each repeating unit i may have different residues for each [An] and [Ac] but all repeating units i have [Ag] with identical residues.

In a second embodiment, a fibril forming peptide is provided. The peptide has a primary structure given by:

f_{N}-[An₁]-(Gly-X-Y)ₙ-[Ac₁]-L-[An₂]-(Gly-X-Y)ₙ-[Ac₂]-L-f_{C}

wherein the (Gly-X-Y)ₙ is a three-residue amino acid sequence wherein every third residue in the (Gly-X-Y)ₙ is Gly; n is an integer such that 2≤n≤70; f_{N} is an N-terminal overhang region having between 9 and 50 amino acids; f_{C} is an C-terminal overhang region having between 9 and 50 amino acids; L is a linker given by (Gly-Pro-Z)ⱼ where j is an integer such that 2≤j, Z is Pro or Hyp; [An₁], [An₂][Ac₁] and [Ac₂] are independently selected chains of 0-3 amino acid residues.

In a third embodiment, a fibril forming peptide is provided. The peptide has a primary structure given by:

f_{N}-(Gly-X-Y)ₙ-[Ac]-L-(Gly-X-Y)ₙ-L-f_{C}

wherein the (Gly-X-Y)ₙ is a three-residue amino acid sequence wherein every third residue in the (Gly-X-Y)ₙ is Gly; n is an integer such that 2≤n≤70; f_{N} is an N-terminal overhang region having between 9 and 50 amino acids; f_{C} is an C-terminal overhang region having between 9 and 50 amino acids; L is a linker given by (Gly-Pro-Pro)₄ (SEQ ID NO: 4); [Ac] is a chain of 0-3 amino acid residues.

This brief description of the invention is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings are not necessarily to scale, emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings in which:

FIG. 1 depicts a structure hierarchy of collagens in tissues;

FIG. 2 depicts a sequence architecture of peptides Col108, 2U108 and 1U108;

FIG. 3 depicts a sequence architecture of peptides Col877 and Col108r;

FIG. 4A and FIG. 4B are TEM image of the d-periodic mini-fibrils of Col877;

FIG. 5A and FIG. 5B are TEM images of the non-specific aggregates of Col108r;

FIG. 6 depicts another sequence architecture of peptide Col108, 2U108 and 1U108;

FIG. 7A, FIG. 7B, FIG. 7C and FIG. 7D are TEM pictures of the d-periodic mini-fibrils of 2U108;

FIG. 8 is a TEM image of the 35 nm d-period of 2U108 mini-fibrils;

FIG. 9A, FIG. 9B, FIG. 9C and FIG. 9D are TEM pictures of the non-specific aggregates of 1U108;

FIG. 10A depicts a SDS-PAGE study of peptide 2U108;

FIG. 10B shows formation of non-specific aggregates of 1U108;

FIG. 11A, FIG. 11B, FIG. 11C and FIG. 11D are TEM images showing the aggregates of 2U108 that have the appearance of smooth, mini-fibrils;

FIG. 12A, FIG. 12B, FIG. 12C and FIG. 12D are TEM images showing aggregates of 1U108 that have very different appearances from that of 2U108;

FIG. 13 is a schematic depiction of gap and overlap configurations;

FIG. 14 depicts another sequence architecture of peptide Col108, 2U108 and 1U108.

### DETAILED DESCRIPTION OF THE INVENTION

This disclosure relates to the generation of collagen-mimetic protein fibrils. The fibrils are formed through the lateral self-association of triple helical peptides. The fibrils have the axial repeating structure, designated as the d-period, which is reminiscent of the D-period of fibrillar collagens. The actual size of the d-period, as well as that of the gap or overlap region are among the design features that can be controlled and optimized to meet the needs of specific applications. The self-association process is reversible by nature and is activated by the variation of buffer conditions. However, further cross-linking, including those observed in native collagens, can be engineered to covalently link the triple helices in the fibrils to inhibit the dissociation of the fibrils.

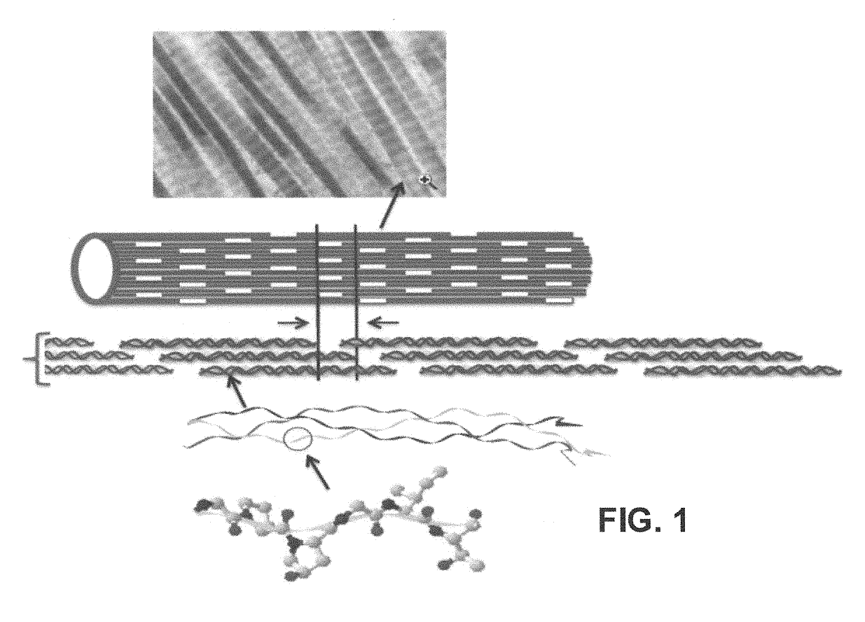

It has been well established that peptides with the Gly-X-Y repeating sequence, where X and Y can be any amino acid residues, will form collagen triple helix. As shown in FIG. 1, the biological functions and properties of collagens are closely related to this structural hierarchy. Starting from the bottom of FIG. 1, a single peptide chain in (Gly-X-Y)ₙ sequence assembles to form a triple helix. Triple helixes form a staggered arrangement of the triple helices in fibrils that have the D-periodic spacing of the fibrils. The top pane shows the D-periodic fibrils in tissues.

As discussed in The Journal of Biological Chemistry, Vol. 290, No. 14, pp 9251-9261, Apr. 3, 2015 in an article entitled “The Self-assembly of a Mini-fibril with Axial Periodicity from a Designed Collagen-mimetic Triple Helix” the peptide Col108 having three repeating sequence units can mimic the lateral association process of collagen triple helices during fibrillogenesis to form mini-fibrils showing d-period like structure. The d-period of the Col108 mini-fibril is related to the periodicity in the amino acid sequence. The triple helix domain of Col108 consists of three pseudo-identical units (i=3) of amino acid sequence arranged in tandem. A mutual staggering of one sequence unit of the associating Col108 triple helices can produce the 35 nm d-period observed by electron microscopy and atomic force spectroscopy.

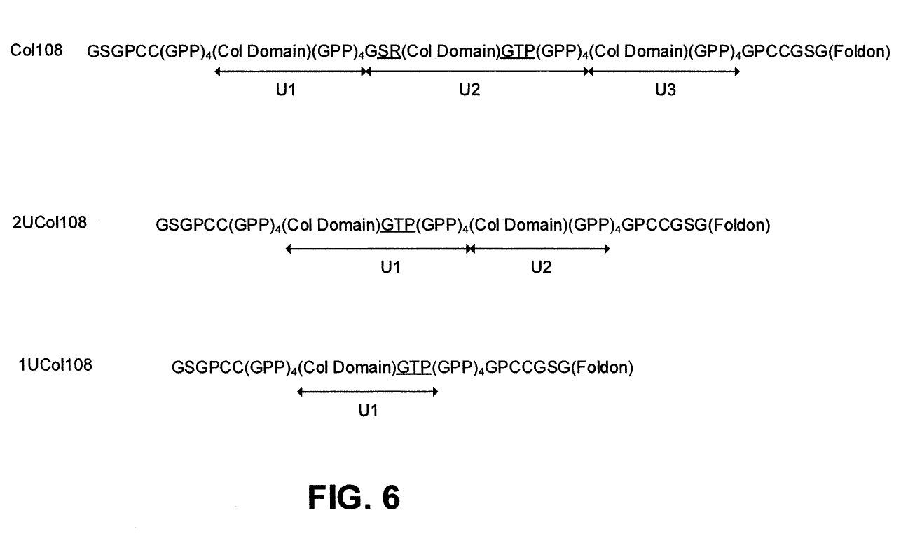

f_{N}-((Gly-X-Y)₃₆-(Gly-Pro-Pro)₄)ᵢ-f_{C} (1)

wherein f_{N} is an N-terminal overhang, f_{C} is a C-terminal overhang, i is a non-zero integer (in Col108, i=3). The sequence (Gly-X-Y)₃₆ consists of 108 amino acid residues and is termed the Col-domain. In the second sequence unit, U2, there are additional insertions of 3 amino acid residue segments at the N-terminal (the [An]), and the C-terminal (the [Ac]) of the col-domain; with [An]=GSR, [Ac]=GTP (FIG. 6). The overhang regions (f_{N} and f_{C}) consist of residues at the N- and the C-termini of the peptide, respectively. The overhang regions do not conform to the Gly-X-Y repeating sequence and are thus, not necessarily in the triple helix conformation.

While Col108 formed fibrils the reason for fibril formation was unclear. The sequence architecture and the specific sequence of the Col-domain (i.e. the extensive repetition of (Gly-X-Y)₃₆) were postulated to be involved in the formation of the d-period mini-fibrils of Col108. However, as discussed in detail below, this postulate was not correct. The discoveries outlined in this disclosure have permitted the development of a new set of design rules that allows one to produce alternative fibril-forming materials that do not rely on the specific residues in the (Gly-X-Y)₃₆ sequence.

The current disclosure pertains to a set of design rules about the organizational requirements to be imposed on the primary sequence of the peptides. The peptides can be generated by solid-phase peptide synthesis or by expression systems using the recombinant DNA technology.

Without wishing to be bound to any particular theory, based on this 1-unit staggering model a triple helix with two sequence units (i=2) is expected to have the potential to form the same d-periodic mini-fibrils.

The sequence of the triple helical domain (the (Gly-X-Y) repeating sequence) is organized into units (Uᵢ) placed in tandem. The units Uᵢ, where i=2 should have highly similar amino acid sequences and identical number of residues in (Gly-X-Y)ₙ repeats, and the linker region Lᵢ: Lᵢ=Gly-Pro-Z)ⱼ, Z=Pro or Hyp, j≥2. Additional short segments of amino acid residues [An] and/or [Ac] may be included in the sequence unit; where each [An] and [Ac]=0-3 amino acid residues (including Hyp and Hyl). In one embodiment, [An] and [Ac] have an identical primary structure. In another embodiment, [An] and [Ac] have different primary structures. The number of residues in the Uᵢ, is chosen to create a fibril having a d-period of the size of approximately [(n+j)×0.9 nm], where 2≤n≤70, j>2. Given 2≤n≤70, the overall length of the sequence (Gly-X-Y)ₙ is between six residues (n=2) and 210 residues (n=70), provided every third residue is Gly. In one embodiment, 2≤j≤6. In another embodiment 2≤n≤40.

f_{N}-([An]-(Gly-X-Y)ₙ-[Ac]-Lᵢ)₂-f_{C} (2)

Each of the overhang regions f_{N} and f_{C} at the N- the C-termini of the peptide, respectively, generally has between 9-50 residues. They can be in any sequence and adopt to any conformation, as long as the structure do not interfere the folding of the triple helix and the fibril assembly.

This disclosure demonstrates two repeating sequence units (i=2) are necessary and sufficient for a designed collagen triple helix to form collagen-mimetic fibrils through lateral self-association. The axial repeating structure (i.e. the d-period) correlates precisely to the size of the sequence unit; the size of the gap or the overlap of the d-period can be controlled by the size of the overhang region(s) of the peptide. The combined size of fully folded f_{N} and f_{C} (any amino acid sequences, in any folded conformations) equals the desired fraction of the d-period—the size of desired ‘overlap’. In some embodiments, more than three repeats (i.e. i>3) is used. Without wishing to be bound to any particular theory, when i>3 more stable fibrils are believed to be formed.

The linker unit (Lᵢ), is given by (Gly-Pro-Z)ⱼ where Z is Pro or Hyp and j is an integer that is greater than or equal to 2.

The additional amino acid residues [An] and/or [Ac] can be included in the sequence unit, where [An] and [Ac] each is a chain of 0-3 residues, each of which may be any amino acid. With the inclusion of the [An] and/or [Ac] sequences, the sequence units are pseudo-identical. Without the insertions, the two units are identical.

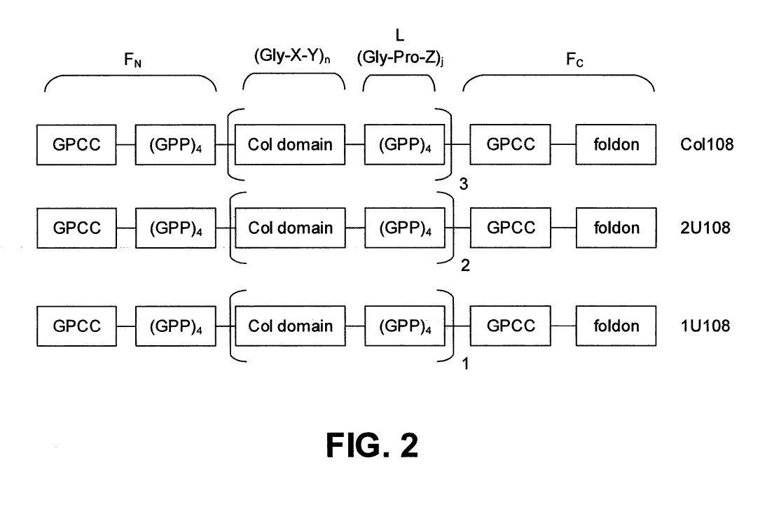

Referring to FIG. 2, when such a peptide (designated 2U108) was made it was found to form mini-fibrils having the same d-period of 35 nm. In contrast, no such d-periodic mini-fibrils were observed for peptide 1U108, which has only one sequence unit. Without wishing to be bound to any particular theory, Applicant believes 1U108 did not produce fibril-like structures because the repeating unit has been omitted (e.g. i is less than 2). All the triple helices are produced from an E. coli expression system using artificial genes. The findings of the periodic mini-fibrils of Col108 and 2U108 suggest a way forward to create collagen-mimetic fibrils for biomedical and industrial applications. In these examples, to create the structural features of gaps and overlaps, the overhang units included have a size equivalent to 0.3 d (about 10 nm) comprised of the N and C-terminal sections of peptide bracketing the three sequence units; specifically, the overhang consists of an N-terminal Cys-knot sequence, a N-terminal (Gly-Pro-Pro)₄ sequence (SEQ ID NO: 4), a C-terminal Cys-knot sequence and a C-terminal foldon domain.

This disclosure also demonstrates that the specific amino acid sequences of the (Gly-X-Y)ₙ domain is of secondary importance.

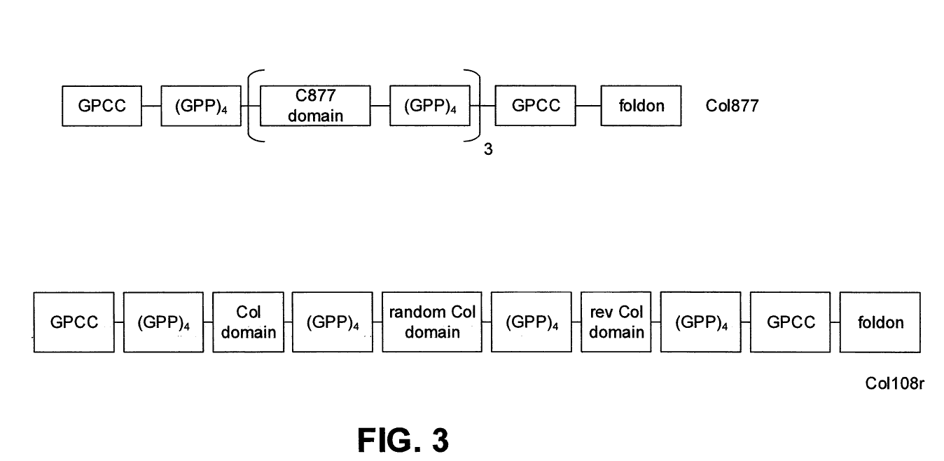

Referring to FIG. 3, the present disclosure also pertains to the self-assembly of peptide Col877. Peptide Col877 has three pseudo-identical sequence units (i=3) placed in tandem, in a fashion similar to that of Col108, except the amino acid sequences of the repeating unit is completely different from that of Col108 and 2U108. Another amino acid sequence, Col108r, has the exact composition as that of Col108 but lacks the periodic placement of key amino acid residues. Both Col877 and Col108r form collagen triple helix with comparable thermal stability. Yet, only the self-assembly of Col877 produced mini-fibrils having the same d-period as that of Col108. Col108r did not form any fibril-like structures with discernable structural features when examined using electron microscope. Without wishing to be bound to any particular theory, Applicant believes Col108r did not produce fibril-like structures because the repeating unit has been omitted (e.g. i is less than 2). The findings of Col877 and Col108r accentuated the desirability of the sequence periodicity; while the actual sequence being repeated is of secondary importance.

FIG. 3 schematically depicts the sequence architecture of Col877 and Col108r. The overall organization of the amino acid sequences of Col877 and Col108r are shown in a block presentation, together with that of Col108 for comparison. The C877 domain consists 108 amino acid residues taking from residues 877-985 of the triple helical domain of the al chain of human type I collagen.

Col108 in FIG. 2 adheres to the equation (1) design as follows: f_{N} is a Cys-knot (GPCC)-(Gly-Pro-Pro)₄(SEQ ID NO: 5); (Gly-X-Y)ₙ is the Col domain; i is 3, and f_{C} is a Cys-knot (GPCC) plus a foldon region. The Col domain consists 108 amino acid residues as follows: GERGPPGPQGARGLPGAPGQMGPRGLPGERGRPGAPGP AGARGEPGAPGSKGDTGAKGEPGPVGVQGPPGPAGEEGKRGARGEPGPTGPAG PKGSPGEAGRPGEAGLP (SEQ ID NO: 1) taken from four selected regions of the al chain of human type I collagen.

2U108 in FIG. 2 adheres to the equation (2) design as follows: f_{N} is a Cys-knot (GPCC)-(Gly-Pro-Pro)₄ (SEQ ID NO: 5); (Gly-X-Y)ₙ is the Col domain; and f_{C} is a Cys-knot (GPCC) plus a foldon region.

1U108 in FIG. 2 violates the equation (2) design as follows: f_{N} is a Cys-knot (GPCC)-(Gly-Pro-Pro)₄ (SEQ ID NO: 5); (Gly-X-Y)ₙ is the Col domain; and f_{C} is a Cys-knot (GPCC) plus a foldon region.

Col877 in FIG. 3 adheres to the equation (1) design as follows: f_{N} is a Cys-knot (GPCC)-(Gly-Pro-Pro)₄ (SEQ ID NO: 5); (Gly-X-Y)ₙ is the C877 domain; and f_{C} is a Cys-knot (GPCC) plus a foldon region. The C877 domain has 108 amino acid residues as follows: GPVGPAGKSGDRGETGPAGPAGPVGPVGARGPAG PQGPRGDKGETGEQGDRGIKGHRGFSGLQGPPGPPGSPGEQGPSGASGPAGP RGPPGSAGAPGKDGLNGLPGPI (SEQ ID NO: 2).

Col108R in FIG. 3 violates the equation (2) design. Specifically, the (Gly-X-Y)ₙ in the three units are not identical. (Gly-X-Y)ₙ in the first unit is the Col-domain, (Gly-X-Y)ₙ in the second unit is the Randomized Col-domain, and the (Gly-X-Y)ₙ in the third unit is the reversed Col-domain. The Randomized Col domain and the reversed Col domain represents, respectively, the randomized placement of the residues of the Col domain and the placement with inversed C-to-N directionality of the residues of the Col domain. In both Randomized Col domain and Reversed Col domain the (Gly-X-Y) sequence pattern of collagen triple helix is maintained. The sequences of the f_{N} overhang, the f_{C} overhang and the foldon domain (GYIPEAPRDGQAYVRKDG EWVLLSTFL, SEQ ID NO: 3) are common features of all five peptides.

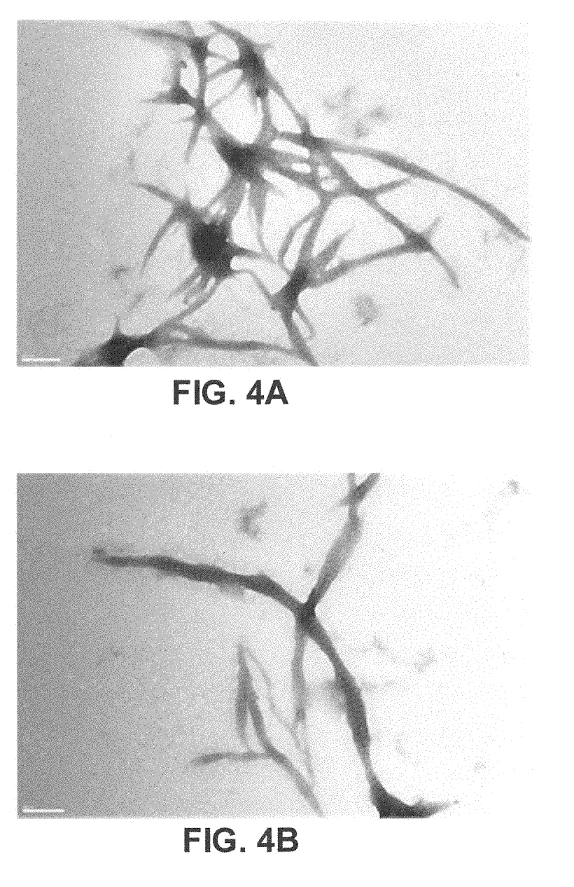

FIG. 4A and FIG. 4B are TEM images of the d-periodic mini-fibrils of Col877. The fibril formation was initiated by transferring Col877 in 5 mM acetic acid (pH 4.5) to TES buffer (pH 7) and incubated for 24 hr at 37° C. The samples were stained with 1% sodium phosphotungstate; magnification factor 10,000; the scale bar is 200 nm. The two images were taken for two different preparations under identical treatments. These figures show Col887 successfully forms fibrils.

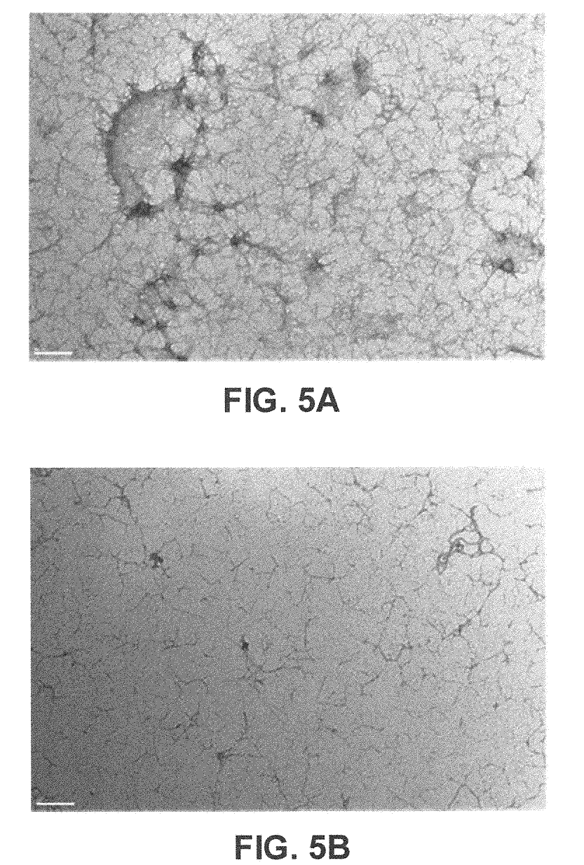

FIG. 5A and FIG. 5B are TEM images of the non-specific aggregates of Col108r. The fibril formation was initiated by transferring Col108r in 5 mM Acetic Acid (pH 4.5) to TES buffer (pH 7) and incubated for 24 hr at 37° C. The grids were stained with 1% sodium phosphotungstate; magnification factor 10,000; the scale bar is 200 nm. The two images were taken for two different preparations under identical treatments. These figures show Col108r did not form fibrils.

FIG. 6 depicts the sequence architecture of peptide Col108, 2U108 and 1U108. The U1, U2 and U3 were the terminology used for, respectively, the three sequence units of Col108. The (Kpnl) marks the location of the restriction enzyme sit kpnl in the genes of the peptides.

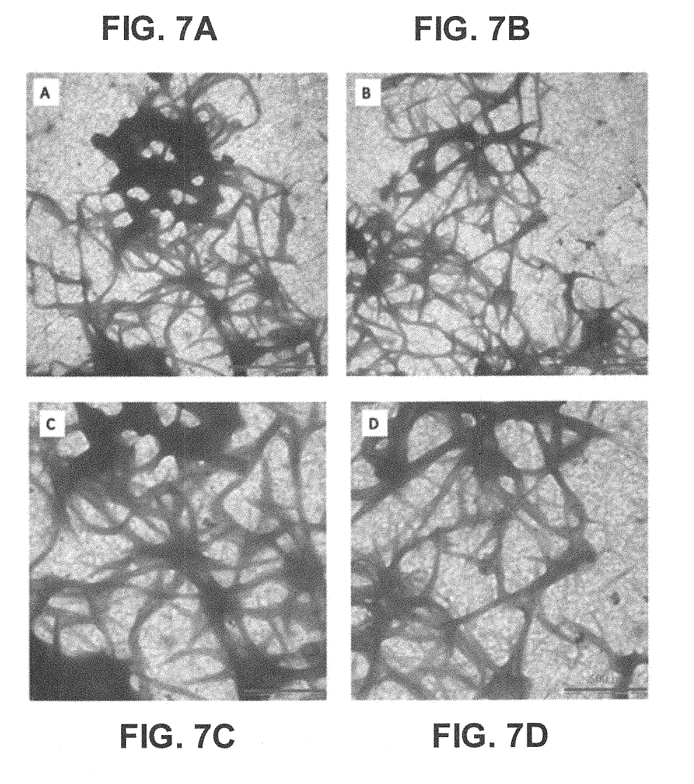

FIGS. 7A to 7D are TEM pictures of the d-periodic mini-fibrils of 2U108. FIG. 7A and FIG. 7B depict negatively stained 2U108 mini-fibrils after 24 h incubation at 37° C. at the magnification of 30000. FIG. 7C and FIG. 7D depict negatively stained 2U108 mini-fibrils after 24 h incubation at 37° C. at the magnification of 50000. These figures show 2U108 successfully formed fibrils.

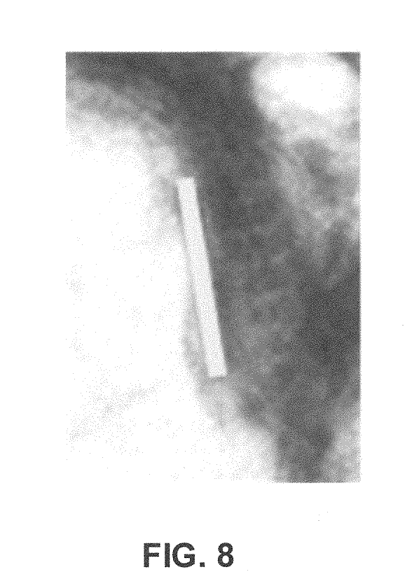

FIG. 8 shows the 35 nm d-period of 2U108 mini-fibrils. The scale bar is 200 nm. The repeating sequence unit of 120 residues leads to the formation of d-period about 35 nm in size, consisting a gap (the dark band, about 20 nm) and an overlap region (light band, about 15 nm).

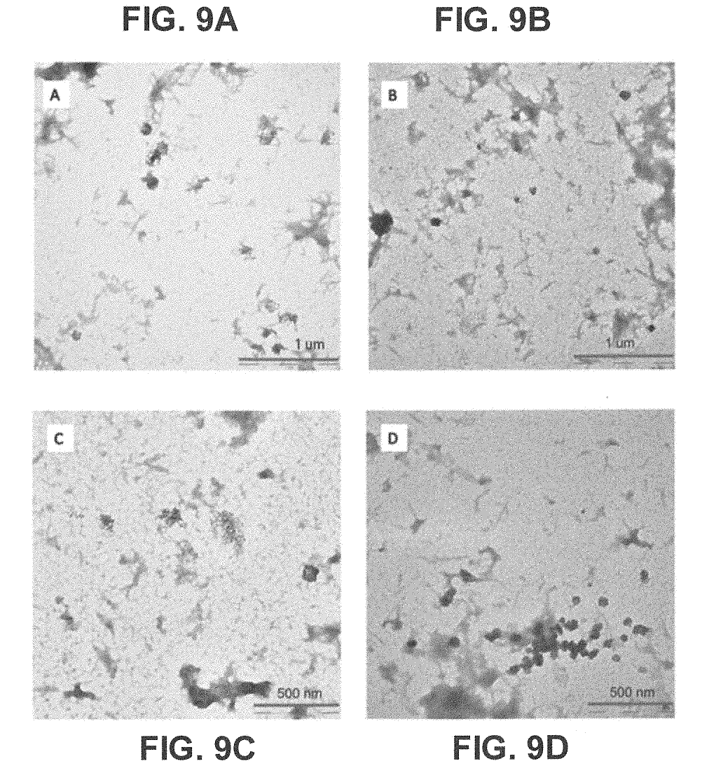

FIGS. 9A to 9D are TEM pictures of the non-specific aggregates of 1U108. FIG. 9A and FIG. 9B are negatively stained aggregates after 24 h incubation at 37° C. at the magnification of 30000. FIG. 9C and FIG. 9D are negatively stained aggregates after 24 h incubation at 37° C. at the magnification of 50000. These figures show 1U108 did not form fibrils.

Collagen-based biomaterials generated using the disclosed bottom-up approach offer a new alternative for cost-effective applications based on collagen. These materials can be made using peptides having specifically selected amino acid sequences and are a fraction of the size of the full-chain collagens. These bottom-up materials have a major advantage—the amino acid sequences of the peptides; thus the functions and the overall properties of the materials can be fine-tuned and optimized for specific applications. For example, triple helical peptides can be designed to carry a particular enzyme recognition sequence to target a specific biological interaction; or the sequence can be optimized to prevent the degradation of the collagenase of the tissues. While making peptides to fold into the triple helix only requires the amino acid sequences to have Gly at every third position, obtaining peptides that can further assemble into fibrils has proven to be challenging. In fact, Col108 is the first mimetic material that can form fibrils. The assemblies of the triple helices other than Col108 generate higher-order molecular structures that are very different from that of the native collagen fibrils, and frequently rely on the incorporation of heavy metal ions and/or non-biological chemical linkages. The dissimilar overall-structures translated to differences in the tensile strength and other properties of such materials from that of natural collagens, and limited the applications of the materials.

This disclosure also demonstrates the self-assembly of the collagen-mimetic fibrils is not limited to the amino acid sequences selected for Col108 and 2U108. Regardless the precise amino acid residues that are being periodically placed in the sequence, as long as the entire amino acid sequence of the peptide is organization into pseudo-identical units placed in tandem, the peptide will form the d-periodic mini-fibrils through self-association. The size of the d-period and that of the gap-overlap regions depend only on the size of the repeating sequence units and of the overhangs of the non-triple helical domains. The precise amino acid residues within the sequence units have little effects on the overall structural features of the d-period.

The application of the disclosed collagen-based material is exceedingly broad. The collagen-mimetic protein fibrils generated using this currently disclosed method can be used in the areas that rely on collagens extracted from animals, collagens from expression systems, or triple helical peptides, including but not limited to the following: Drug-delivery and medical-devices, as soft tissue fillers, cosmeceuticals, molecular scaffolds for tissue regeneration, food industry, industrial use of gelatin, and material Bio-fabrications. The protein-based biomaterials 1) have a molecular scaffold modeling of the D-periodic fibrils of fibrillar collagens and 2) have tunable functions and adjustable sizes for desired applications. The disclosed method provides the capability to produce collagen-mimetic biomaterials that have the tensile strength and the molecular scaffolds comparable to that of native collagen fibrils. Such materials can serve as safer and cheaper alternatives to collagens extracted from animals or be produced from expression systems. The disclosed biomaterials can be incorporated into other protein design strategies to generate materials that utilize the supramolecular structure of collagen-fibrils to achieve desired tensile strength and/or molecular microenvironment for applications. A method for designing the protein fibrils specify the conditions for a designed peptide to 1) fold into the conformation of collagen triple helix and 2) further self-assemble into D-periodic fibrils.

The disclosed materials can be used to replace collagens in the established collagen-related applications, and extend the scope of the new industry of collagen-mimetic materials. The materials are safe for medical related uses, and even easier and cheaper to produce than the recombinant collagens using expression systems. At the same time, the material offers the potential and feasibility of incorporating special design features formulated for specific applications.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

### EXPERIMENTAL

The Triple Helical Peptides 2U108 and 1U108

The primary structures of peptide 2U108 and 1U108 are designed based on the original amino acid sequence of Col108. Different from Col108, the triple helix domain of 2U108 consists of only two sequence units, and that of 1U108 only one (FIG. 2). Each sequence unit is composed of a 108-residue Col-domain, and an C-terminal (Pro-Pro-Gly)₄ linker. The primary structure of all three peptides share a few common features including the N-terminal (Pro-Pro-Gly)₄ sequence, the Cys-knot sequences at the N- and C-termini of the triple helix domain, and the C-terminal foldon domain. The foldon is included to ensure proper folding of the triple helix, and the Cys-knot to increase the stability of the triple helix conformation through a set of inter-chain disulfide bonds. Similar to Col108, the tripeptide (Gly-Thr-Pro) insert between the two sequence units of 2U108 is due to the use of a Kpnl restriction enzyme site in the gene. In all, peptide 2U108 has 295 residues, with 255 of them being in the triple helix domain and having a non-interrupted Gly-X-Y repeating sequence; peptide 1U108 has a total of 172 residues encompassing a 132-residue triple helix domain. As in the case of Col108, the amino acid residues in 2U108 have a periodic placement because of the repeating sequence units—the residues in the Col domain are being repeated after 123 residues. In contrast, none of the residues of 1U108 have any long-range periodic placement.

Peptide Col877 and peptide Col108r are generated using similar methods, except the genes were synthesized using commercial services based on the provided gene sequences.

The CD spectra of both 2U108 and 1U108 in 5 mM acetic acid (pH 4) are indicative of a triple helix conformation, which is characterized by a deep negative peak at 197 nm and a small positive peak at 225 nm. The Rpn values (the ratio of the two peaks) of about 0.09 for both peptides are comparable to that of a typical triple helix conformation having a high content of Gly-Pro-Pro sequences but no Hyp. The triple helix conformation of both peptides is quite stable despite lacking the stabilizing Hyp in the Y-positions; the melting temperature of both peptides is about 41° C. The foldon domain, the Cys-knots at both ends of the triple helical domain, and the high content of charged residues may all contribute to the stability. Despite the significant differences in size, the melting temperatures of U2108 and 1U108 are in good agreement with that of Col108. Similar length-independent thermal-stability has also been observed in studies of bacterial collagens. The CD spectra of Col877 and Col108r are similar to that of 2U108 and 1U108, except the melting temperature of Col877 is 40° C. and that of Col108r is 39° C. due to the variations in the amino acid sequence.

The self-association of 2U108 and 1U108 in TES buffer:

Fibrillogenesis of collagen in vitro, and to an extent also in vivo, is a process mediated by electrostatic interactions; triple helices obtained from acid-dissolved tissues in cold temperatures will spontaneously self-associate into fibrils once the pH is increased to about 7, and the temperature increased to the range of physiological temperatures, usually 25-37° C. The self-assemblies of 2U108, 1U108 Col877 and Col108r were studied following the same in vitro fibrillogenesis procedure of native collagen. All samples were equilibrated in the refrigerator in pH 4 buffer for at least 48 hr to ensure proper folding. The fibril formation was initiated by raising pH and temperature. The self-association in the fibril-forming buffer was monitored using a modified SDS-PAGE experiment. In this approach, in addition to the standard denaturation procedure utilizing SDS, boiling and the addition of reducing agent, the peptide samples were prepared using two other denaturation conditions. The mild denaturation condition, which includes the addition of SDS but no boiling and no addition of reducing agent, is devised to maximally preserve the aggregates by minimizing the disruptions of the non-covalent interactions and the unfolding. The non-reducing condition includes SDS and boiling but without the addition of reducing agent. Under this condition, all non-covalent interactions stabilizing the triple helices and the aggregates will be maximally disrupted, but the structures related to disulfide bonds will be preserved. This non-reducing condition can effectively probe the involvement of the disulfide bonds during the self-association of the triple helix.

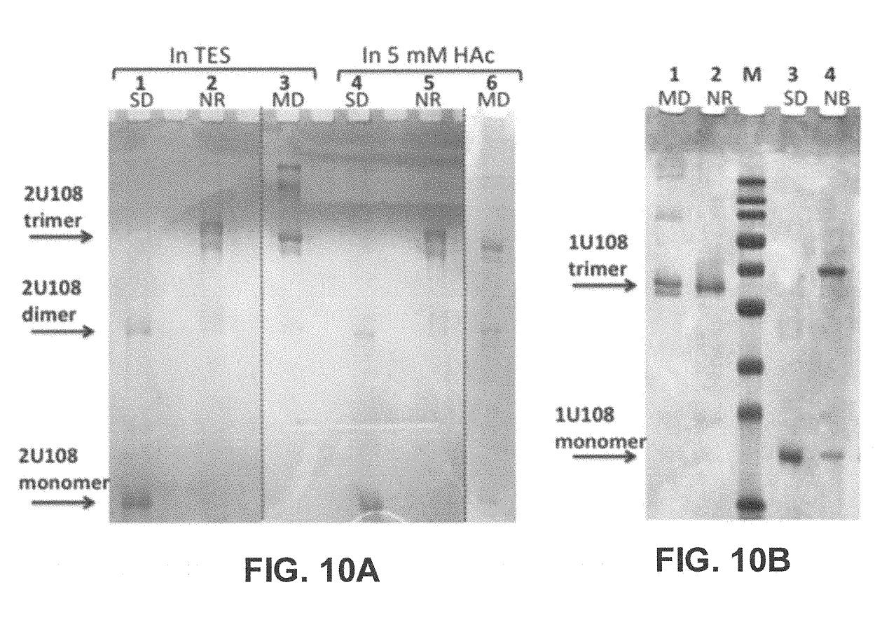

The SDS-PAGE studies of peptide 2U108 are shown (FIG. 10A). Under mild denaturation, the triple helices with fully formed Cys-knots at the N- and/or C termini are expected to migrate as a trimer on the gel; any self-assembled species formed during incubation in the fibril-forming (TES) buffer will appear as species with molecular weight higher than that of a trimer. Indeed, significant amount of aggregates of 2U108 are preserved and can be observed as bands with molecular weights higher than that of the peptide trimer (FIG. 10A, Lane 3). After boiling but without the addition of the reducing agent (the non-reducing condition), only the trimer form of 2U108 was observed (FIG. 10A Lane 2). The complete disappearance of bands of high molecular weight species under the non-reducing condition indicate the self-assembled species of Lane 3 were stabilized by non-covalent interactions between the triple helices without the involvement of the inter-helical disulfide bonds. Finally, the addition of reducing agent (the standard denaturation condition) abolished the intra-helical disulfide bonds, and completed the unfolding of the triple helices and the aggregates into monomers (FIG. 10A, Lane 1). Since the Cys-knots are known to form only in the structural context of a fully folded triple helix, the lack of any significant presence of dimer or monomer in lane 2 and lane 3 is a strong indication that the 2U108 peptide is in the triple helix form before the initiation of fibril formation. The trimer form of 2U108 often resolves into multiple bands on a gel under the non-reducing condition due to the compounded effects of the rodshape of the triple helix conformation and the cross linking of the inter-chain disulfide bonds at the ends. The fully folded, trimeric 2U108 has a large aspect ratio: 150 nm long and only 1-2 nm in diameter. With the inter-chain disulfide bonds intact, some of the partially unfolded triple helices may retain a significant level of the rod-shape, and behave differently from the coiled form of the unfolded molecules during the electrophoresis. The multiple trimer bands reflect the different mobilities of a heterogeneous population of partially folded triple helices varying in degrees of ‘foldedness’ and compactness. Without the complications of shape and residual structures, the monomers in Lane 1 emerge as a single, clearly defined band on the gel. A small amount of dimer is often present despite extensive boiling in the presence of reducing agent(s) (about 45 min). Each Cys-knot can potentially form three inter-chain disulfide bonds in a folded triple helix; the complete reduction of the multiple disulfide bonds proves to be difficult.

In contrast, no bands of molecular weights higher than that of the trimer were observed under the mild denaturation condition for the original 2U108 sample in HAc buffer before fibril formation (FIG. 10A, Lane 6); the trimer band(s) appeared to be the major species present with a trace amount of dimer. Similarly, only trimers were observed under the non-reducing condition (FIG. 10A lanes 5), and only monomers are found in the presence of the reducing agent (FIG. 10A lane 4). A portion of the sample loading wells is kept in the gel pictures to check on the possible presence of any aggregates that might be too large to enter the stacking gel and/or the separation gel.

The 1U108 molecule appears to form aggregates as well as shown in FIG. 10B. Several bands with molecular weights higher than that of the trimer are clearly observable under the mild denaturation condition (FIG. 10B, Lane 1). The aggregates are reversibly returned to trimers by boiling under the non-reducing condition (FIG. 10B Lane 2); and only the monomer form is present under the standard denaturation condition (FIG. 10B Lane 3). Interestingly, a large portion of the denatured peptide migrated as a trimer in the presence of reducing agent but without boiling (FIG. 10B, Lane 4). The residue structure of the triple helix under this condition was suspected to prevent full reduction of the disulfide bonds of the Cys-knots.

In summary, the SDS-PAGE results clearly demonstrated the self-association of both 2U2108 and 1U108 upon incubation in the fibril-formation buffer. These aggregates do not involve disulfide bonds, and are reversible under non-reducing conditions. It needs to be pointed out that, while effective, the SDS-PAGE approach only provides qualitative information on the aggregation. It is impossible to infer the degree of self-association of the peptides based on this approach alone. The presence of SDS alone can cause considerable dissociation of the aggregates. The actual amount of the aggregates could be significantly greater than what is indicated by the number and density of high molecular weight bands. Neither can this technique provide any information on the size or shape of the aggregates.

The characterization of the self-assembled aggregates:

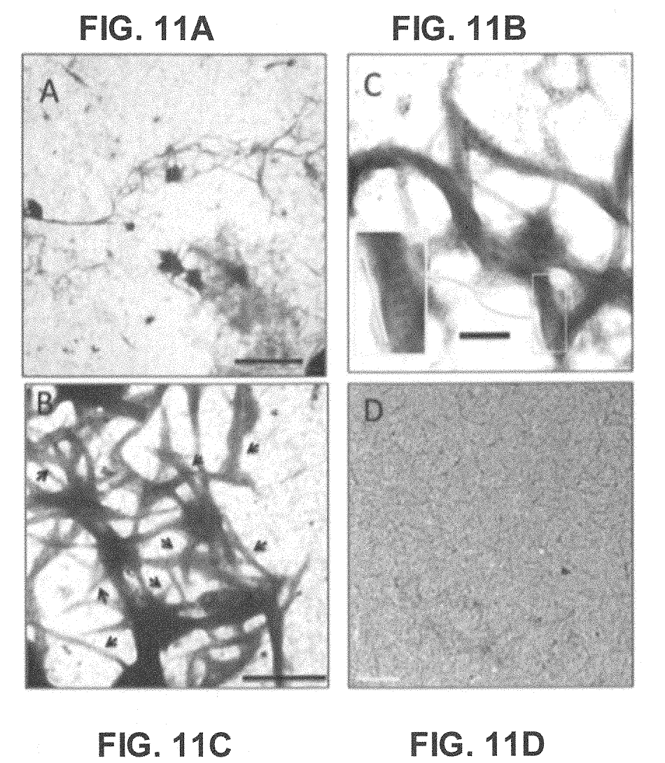

The size, shape and structural features of the aggregates of 1U108 and 2U108 in fibril-forming buffer were examined using transmission electron microscopy (TEM). As shown in FIG. 11A-11D, the aggregates of 2U108 have the appearance of smooth, mini-fibrils with tipped ends, similar to those observed for Col108. The minifibrils are about 500 nm-1 μm in length, with the diameters of the central part of the mini-fibrils varying between about 20 nm to about 75 nm. The tipped ends are characteristic of collagen fibrils formed by lateral association of the triple helices. A closer examination of the negatively stained mini-fibrils of 2U108 revealed the recognizable striated banding pattern of about 35 nm, consisting of a dark band of about 25 nm and a white strip of about 10 nm (FIG. 11B and FIG. 11C). This banding pattern resembles those of collagen and Col108 in appearance, and agrees with the d-period of Col108 minifibrils in size.

The mini-fibrils are formed only after being transferred into the pH7 buffer. The TEM image of 2U108 in HAc reveals a striking contrast, showing a uniform background of 2U108 triple helices with no mini-fibrils (FIG. 11D). The thread looking ‘structures’ appear to be 2U108 monomers (triple helices) judging by their size (expected triple helix about 85 nm in length, about 1-2 nm in diameter). No large aggregates were observed. The inhibition of fibril formation in pH 4 buffer is also known for native collagens. The low pH condition can, presumably, disrupt the electrostatically driven interactions between the helices by promoting the protonation of acid residues. The sensitivity of the self-assembly of 2U108 to pH thus supports the view that the mini-fibrils form by a mechanism similar to that for the formation of native collagen fibrils.

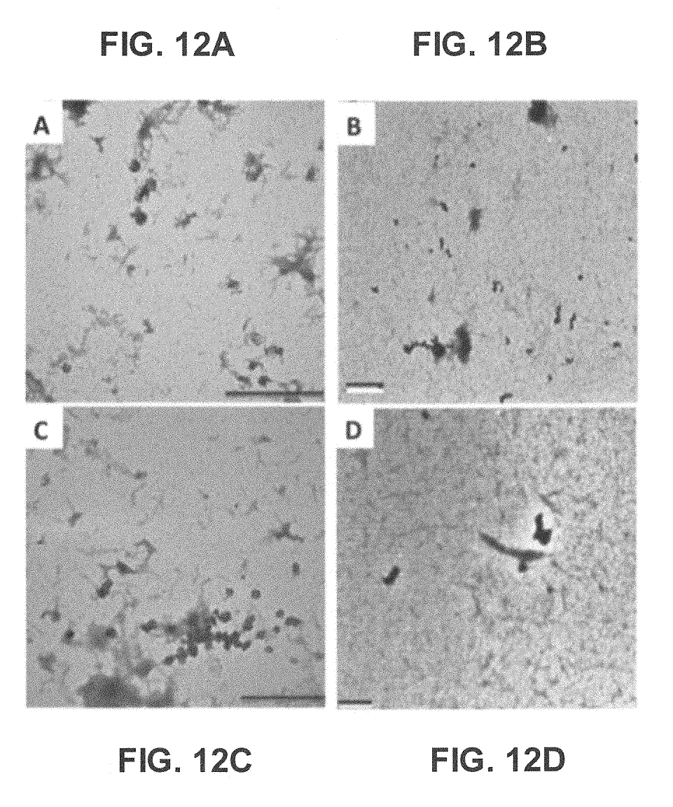

The aggregates of 1U108 in pH7 buffer, on the other hand, have a very different appearance from that of 2U108 (FIG. 12A to 12D). A 1U108 triple helix is expected to have a length of about 50 nm, and be about 1-2 nm in diameter. There appears to be a considerable degree of self-association to form aggregates up to about 200 nm in length or, occasionally, quite a bit larger (FIG. 12D). Most of the aggregates have an elongated shape with a high aspect ratio. They are likely formed by lateral association of the triple helices, but do not follow any specific pattern. There is no discernable banding pattern or other structural features—though this observation is limited by the resolution of the TEM and staining technique. The self-associated species of 1U108 is therefor considered to be non-specific aggregates.

### DISCUSSION

The 2U108 mini-fibrils have the same d-period as that of Col108 mini-fibrils formed under the same conditions. The self-assembly of 2U108 mini-fibrils was anticipated to follow the same unit-staggered mechanism. Two specific factors were identified that work synergistically to make this unit-staggered arrangement the unique, most stable conformation emerging from the self-assembly of Col108: the optimal alignment of interacting residues of associating helices and the reiteration of interactions of these interactions through the repeating sequence units. The similar sequence architecture of 2U108 to Col108—the tandem repeats of the same sequence unit—suggests the same stabilizing factors will also be present during the self-assembly of 2U108. The two factors are also present during the self-assembly of Col877, although the interaction involved a different set of amino acid residues—those of the C877 domain. Because of the differences in the sequences between the Col-domain and the C877 domain, both the nature and the extent of the stabilizing interactions are different. The self-assembly of the d-periodic fibrils indicate the sequence architecture are the dominating factures for the formation of the d-periodic fibrils.

The stabilizing interactions of 2U108 and Col108 mini-fibrils come from residues in the Col-domain and, to a smaller extent, from the foldon domain. Regions having high content of hydrophobic residues, as well as clusters of charged groups can be readily identified from the amino acid sequence of the Col-domain. In a unit-staggered arrangement, these residues will be placed in the close vicinity of comparable residues from the neighboring helices, which promotes the stabilizing interactions. The residues on the surface of the foldon domain can potentially interact with the neighboring triple helices and contribute to the stability of the fibril assembly. These foldon interactions, however, are limited in extent and are not considered a deterministic factor for the self-assembly process of the mini-fibrils. This situation is further demonstrated by the lack of any d-periodic mini-fibrils for 1U108. Having the same foldon domain and Col-domain, any interactions involving foldon in the self-assembly of Col108 and 2U108 mini-fibrils are available for the self-association of 1U108. Yet, no d-periodic mini-fibrils are observed for 1U108. Lacking periodicity in the primary sequence to direct the specific, staggered assembly of the triple helices, the 1U108 interactions only lead to non-specific aggregates.

For a specific structure to emerge from a self-association, or a folding, process, there must be a stabilization bias toward the specific set of molecular interactions for the desired conformation. The size of a triple helix has profound effects on the self-association of the triple helix because it is directly linked to the number of available interacting residues. In studies of bacterial collagens, it was suggested that the limited self-association of a bacterial collagen variant with a size about ⅕ the length of human fibrillar collagen triple-helix is due to its limited size; an increase in its length may promote the self-association. The triple helix of 1U108 is only about 1/10 the length of a human fibrillar collagen. Yet, there appears to be sufficient molecular interactions between the helices, albeit not in a conformation specific way, to cause aggregation. More than the insufficient size, the lack of any 1U108 mini-fibrils is likely due to the absence of a design element favoring the d staggered self-association over other possible conformations. The contact area between two adjacent 2U108 triple helices in the unit-staggered mini-fibril is more or less the size of a 1U108 molecule. Because of the tandem sequence units of the primary structure of 2U108, such interactions can propagate to other associating helices in the unit-staggered assemblage, and ultimately make the d-periodic minifibrils the most stable conformation to arise from the process.

A successful design strategy often needs to include a mechanism to weaken other potential competing, or miss-folded, conformations. Slightly bulkier foldon domain at the C-terminus may play a critical role in this regard by inhibiting end-on-end stacking of triple helices during the self-assembly. The end-on-end stacking, also referred to as the in-register stacking, represents a conformation with the maximum alignment of the interacting residues, and should therefore be the one that has the highest extent of interaction and thus, the highest stability. Such a structure was not observed in any of the three peptides studied. The tightly packed, trimeric, beta-hairpin propeller conformation of the foldon has a diameter about 25 Å, which is quite a bit larger than that of a triple helix (about 15 Å). The lack of the end-on-end stacking conformation was attributed to the steric hindrance of the bulkier foldon domain at the C-terminal end during the self-assembly. A full understanding of how this bulky structure of the foldon is accommodated in the smooth fibrils of Col108 and 2U108 would require more high resolution structural studies of the mini-fibrils. A close examination of the structure of the foldon suggests that the effects of its bulkiness may be alleviated somewhat by its unique shape. Viewed by the 3-fold symmetry axis of the foldon that is aligned with the axis of the triple helix, the foldon conformation has three slightly concaved faces, perfect for a snugging fit of a triple helix. This close packing of triple helices on the curved surfaces of the foldon is believed to provide a way for the mini-fibrils to circumvent the steric constrains of the foldon. Nevertheless, the bulker size of the foldon domains inside the mini-fibrils may still cause steric tension, and can potentially destabilize and/or limit the growth of the fibril assembly. For future applications, it may prove advantageous or even necessary to remove the foldon domain in the development of collagen-mimetic fibrils. The removal of the foldon from the current construct of Col108 and/or 2U108 can be achieved by including an enzyme digestion site between the foldon and the triple helix domain. However, in the place of a foldon, a new design feature would need to be developed and included to prevent the end-on-end stacking of the triple helices during the self-assembly.

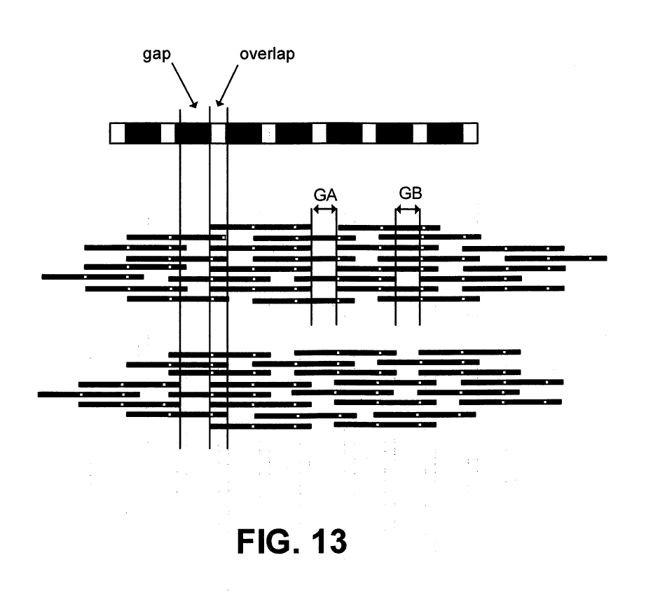

The conformational uniqueness of the mini-fibrils is characterized by the d period—the periodic axial spacing of the gaps and/or the overlaps. The structural characterization based on TEM, using both negative staining and positive staining, and AFM only offers limited resolution on the 3-dimensional structure of the mini-fibrils. The gap regions of the mini-fibrils, as well as that of fibrillar collagens, usually appear as a continuous dark band wrapping around the fibril on negatively stained TEM images. The resolution of TEM leaves other structural details of the region unresolved. There are apparently different ways of packing the unit staggered 2U108 triple helices into mini-fibrils while retaining the 35 nm axial spacing of the gap. As shown in the 2-dimensional presentations in FIG. 13, going transversely across the mini-fibrils in the regions marked GB and GA, the gaps are about 4-5 triple helices apart in GB, but are much closer together in GA—separated by only one triple helix at times. Given the diameter of a triple helix of about 1.5 nm, and the resolution of TEM of about 5 nm, both GA and GB would look like a 25 nm dark band across the whole mini-fibril when examined by TEM. These mini-fibrils with different packings would reveal the same d-period: dark bands every 35 nm intercalated with white strips of about 10 nm. Although the unit-stagger between neighboring triple helices is preserved in all the arrangements, the different packing will generate mini-fibrils with nonuniform distributions of the gaps. It is not clear how the different packing may affect the relative stability of the mini-fibrils. Unless one particular organization is significantly more stable than the others, the self-assembly of 2U108 is likely to be a mixture of mini-fibrils with different distributions of gaps. How such variation in the distribution of gaps affects the other properties of the mini-fibrils remains an interesting question to be fully explored.

The approach of developing d-mini-fibrils using the designed strategy utilized for triple helices Col108 and 2U108 is quite robust. A peptide having the three Col-domains of Col108 replaced by another domain consisting of different amino acid residues also formed d-periodic fibrils, having essentially the same structural features as that of the Col108 and 2U108 mini-fibrils. The d-periodic fibrils of Col877 further demonstrate that the optimal alignment and the reiteration of the interactions of the sequence units will lead to the formation of stable d-periodic fibrils, regardless of the actual sequences in the sequence units. The mini-fibrils and the design strategy presented here will lead to the development of new biomaterials for a broad range of applications.

### CONCLUSION

The identical d-period of 2U108, Col877 and Col108 mini-fibrils indicates a similar molecular recognition process during the self-assembly of the molecules, which mirrors the similarities in their primary structures. The unit-staggered model can explain both the size of the d-period and that of the gap and the overlap regions of the mini-fibrils: the d-period is determined by the size of the sequence unit, and the 0.3 d overhang unit contributes to the overlap region. The specific self-assembly of the mini-fibrils is ultimately determined by the optimization of non-covalent interactions of the associating helices; no inter-helical disulfide bonds or other covalent bonds are involved. The interactions of the residues on the surface of the helices stabilize the self-assembly, while the tandem repeats of the sequence unit determine the structural specificity of the d-period by prescribing a unique way to maximize those interactions. Without such an explicitly designed stability-bias, the self-association of triple helices of 1U108 and of Col108r only led to non-specific aggregates, despite having the same interacting residues. The fibril forming process of 2U108, Col877 and Col108 share the same sensitivity to pH and temperature as that of native collagen fibrils, indicating the same kind of molecular interactions are involved in the self-assembly process. The periodic mini-fibrils of the three triple helices demonstrate the robustness of tandem repeats of sequence units as a design strategy for collagen mimetic biomaterial.

Material and Methods

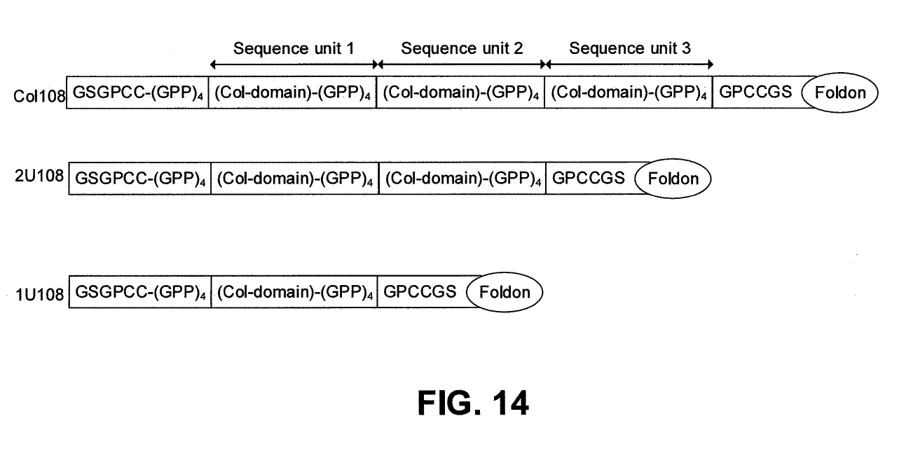

The gene constructs of 2U108 and 1U108-2U108 and 1U108 were created by modifying the original Col108 plasmid. To construct the 2U108 plasmid, a KpnI cleavage site was introduced between the first and the second coding sequences of the Col108 plasmid by affecting a CCA→ACC base change by site-directed mutagenesis (FIG. 14). Together with an existing Kpnl site, the two Kpnl sites bracket the second sequence unit of Col108. The parent plasmid was then digested with KpnI, and the fragments were ligated, resulting in the effective removal of the second sequence unit. The full 2U108 expression plasmid produces a 40.9 kDa fusion protein with a His-tagged thioredoxin attached to the N-terminus of the modified 2U108 peptide, separated by a thrombin cleavage site.

Similarly, the 2U108 plasmid construct was used as the starting point to produce the 1U108 plasmid. A KpnI cleavage site was introduced between the second sequence unit and the C-terminal (GPP)4 (SEQ ID NO: 4) coding sequence by affecting a CCTG-7 TACC base change by site-directed mutagenesis (FIG. 14). The parent plasmid was then digested with KpnI, and the fragments ligated, resulting in the removal of a sequence unit. The full 1U108 expression plasmid produces a 30 kDa fusion protein.

Expression and Purification—the 2U108 and 1U108 peptides were expressed in bacterial strains JM109(DE3) or BL21(DE3). The translation was induced by 0.2 mM IPTG once the OD (600 nm) reached 0.5-0.6 AU. The expression products for 2U108 and 1U108 plasmids were purified using the protocol previously reported. The final product for 2U108 has a molecular weight of 27.1 kDa and is comprised of a triple helix domain containing of two tandemly repeating sequence units with a nucleation sequence, and a C-terminal foldon domain. The molecular weight of peptide 1U108 is 16.2 kDa, comprised of a triple helix domain of a singular sequence unit with a nucleation domain and a C-terminal foldon domain (FIG. 14). Peptides were stored as lyophilized powder at 4° C. until use. Stock solutions were made by dissolving the lyophilized powders in 5 mM acetic acid (HAc), pH 4.0, at a concentration of about 1 mg/mL. The concentration was estimated using extinction coefficients of 0.32 and 0.54, respectively, for 1 mg/mL solutions of 2U108 and 1U108 at 280 nm, calculated using the online tool Protparam.

The characterization of the triple helix conformation—the triple helix conformation of 2U108 and 1U108 were assessed via Circular Dichroism (CD). CD (Aviv Biomedical Spectrometer model 202-01) wavelength scans were conducted at 4° C. between 180 and 300 nm on 0.5 mg/mL peptide samples in the corresponding buffers. Temperature melt experiments were conducted on 0.5 mg/mL peptide samples monitored at a wavelength of 225 nm, and covered a temperature range from 4° C. to 65° C. with an equilibration time of 2 min at each temperature, effectively conferring a heating rate of 0.3° C./min. To aid in the comparison of melt curves between samples, the data was normalized and is displayed in terms of fraction folded, F(T):

$$F ( T ) = \frac{θ ( T ) - {θ}_{uf} ( T )}{{θ}_{f} ( T ) - {θ}_{uf} ( T )}$$

where θ(T) is the observed ellipticity at temperature T, and θ_{f}(T) and θ_{uf}(T) are the ellipticity of the folded and the unfolded triple helix, respectively. The θ_{f}(T) and θ_{uf}(T) were determined from the linear extrapolation of, respectively, the native and the unfolded baselines of the melting curve. The apparent melting temperature is determined as the mid-point of the transition, where F(Tₘ)=0.5.

Fibrillogenesis—To induce fibrillogenesis samples at ˜1 mg/mL, previously dissolved and equilibrated in HAc buffer at 4° C., were mixed with an equal volume of double strength neutralization buffer (60 mM TES, 60 mM Na₂HPO₄, and 135 mM NaCl, pH 7.4) pre-cooled to 4° C. Mixing was conducted on ice, and then the samples were immediately transferred to a water bath set at 37° C. The final concentration of peptide was 0.5 mg/mL and the final composition of the fibrillogenesis buffer after mixing was 2.5 mM acetic acid, 30 mM TES, 30 mM Na₂HPO₄, and 67.5 mM NaCl, pH 7.4 (I=0.09), herein referred to as fibril-forming buffer. The fibrillogenesis samples were tested for fibrils after being incubated for 24 hrs at 37° C.

Electrophoresis—modified SDS-PAGE techniques were used to monitor the self-association of 2U108 and 1U108 in solution, and to test the purity of the samples. The standard denaturation condition was carried out following the standard protocol: 50 μL of sample at 0.5 mg/mL were mixed with 12.5 μL 5×SDS (5%) containing 0.2 M DTT or 2% β-Mercaptoethanol, or both, and boiled for about 45 min in partially sealed eppendorf vials. For denaturation under the non-reducing condition, the samples were prepared using the standard protocol but without the addition of any reducing agent. A mild denaturation condition was devised to denature the peptide by the addition of 2% SDS solution only: the samples did not contain any reducing agent, and were not subjected to heat denaturation (no boiling). In some of the experiments, the samples were prepared following the standard procedure but without boiling; this non-boiling (but reduced) condition was used to test the effectiveness of the reduction of the inter-chain disulfide bonds by reducing agent.

Electron microscope sample preparation—2U108 or 1U108 samples were prepared on 400 mesh formvar carbon-coated copper grids. Three microliters of incubated sample were deposited onto the grids and allowed to sit 100 seconds. The grids were then washed with deionized water by submersing the grids into water for 5 seconds. Immediately following this, 3 μL of a 1% sodium phosphotungstate solution, the staining agent, were applied to the grid and allowed to sit for 100 seconds. The grids were then washed again with deionized water in the previously indicated manner. The grids were air-dried overnight before being examined via electron microscopy (JEM-2100, Jeol Inc.).

Molecular model building—the 3D structures of the triple helix and the foldon domain were generated using the program spdbv. The coordinate files for PDB ID 1RFO and PDB ID 1BKV (triple helical peptide T3-785), for the foldon and the triple helix structures, respectively, were downloaded from RCSB PDB. To create the structural model of a section of the Col-domain, the residues of the T3-785 triple helix were modified to those of the Col-domain, followed by energy minimization after each substitution.

The genes of Col877 and Col108r are synthesized by GenScript. The genes sequences were provided based on the design of the peptides. The expression, purification and characterization of the two peptides follow the same experimental procedures described for 2U108 and 1U108.

What is claimed is:

1. A fibril forming peptide having a primary structure given by:
f_{N}-([An]-[Ag]-[Ac]-Lᵢ)ᵢ-f_{C}
wherein the [Ag] is an amino acid sequence having between 6 and 200 residues wherein every third residue in the [Ag] is Gly;
wherein i gives a number of repeating units such that i=2 or i>3;
f_{N} is an N-terminal overhang region having between 9 and 50 amino acids;
f_{C} is an C-terminal overhang region having between 9 and 50 amino acids;
L is a linker given by (Gly-Pro-Z)ⱼ where j is an integer such that 2≤j, Z is Pro or Hyp; and
[An] is a chain of 0-3 amino acid residues, [Ac] is a chain of 0-3 amino acid residues;
wherein each repeating unit i may have different residues for each [An] and [Ac] but all repeating units i have [Ag] with identical residues.

2. The fibril forming peptide as recited in claim 1, wherein i>3.

3. A fibril forming peptide having a primary structure given by:
f_{N}-[An₁]-(Gly-X-Y)ₙ-[Ac₁]-L-[An₂]-(Gly-X-Y)ₙ-[Ac₂]-L-f_{C}
wherein the (Gly-X-Y)ₙ is a three-residue amino acid sequence wherein every third residue in the (Gly-X-Y)ₙ is Gly;
n is an integer such that 2≤n≤70;
f_{N} is an N-terminal overhang region having between 9 and 50 amino acids;
f_{C} is an C-terminal overhang region having between 9 and 50 amino acids;
L is a linker given by (Gly-Pro-Z)ⱼ where j is an integer such that 2≤j, Z is Pro or Hyp;
[An₁], [An₂][Ac₁] and [Ac₂] are independently selected chains of 0-3 amino acid residues.

4. The fibril forming peptide as recited in claim 3, wherein f_{N} comprises GPCC.

5. The fibril forming peptide as recited in claim 3, wherein f_{N} comprises GPCC(GPP)₄ (SEQ ID NO: 5).

6. The fibril forming peptide as recited in claim 3, wherein f_{C} comprises GPCC.

7. The fibril forming peptide as recited in claim 3, wherein f_{C} comprises GPCC and a foldon sequence.

8. The fibril forming peptide as recited in claim 3, wherein [An₁], [An₂], [Ac₁] or [Ac₂] comprises at least one Hyp residue.

9. The fibril forming peptide as recited in claim 3, wherein [An₁], [An₂], [Ac₁] or [Ac₂] comprises at least one Hyl residue.

10. The fibril forming peptide as recited in claim 3, wherein Z is Pro.

11. The fibril forming peptide as recited in claim 3, wherein 2≤j≤6.

12. The fibril forming peptide as recited in claim 3, wherein j is 4.

13. The fibril forming peptide as recited in claim 3, wherein Z is Pro and j is 4.

14. The fibril forming peptide as recited in claim 3, wherein (Gly-X-Y)ₙ comprises at least one Hyp residue.

15. The fibril forming peptide as recited in claim 3, wherein (Gly-X-Y)ₙ comprises at least one Hyl residue.

16. A fibril forming peptide having a primary structure given by:
f_{N}-(Gly-X-Y)ₙ-[Ac]-L-(Gly-X-Y)ₙ-L-f_{C}
wherein the (Gly-X-Y)ₙ is a three-residue amino acid sequence wherein every third residue in the (Gly-X-Y)ₙ is Gly
n is an integer such that 2≤n≤70;
f_{N} is an N-terminal overhang region having between 9 and 50 amino acids;
f_{C} is an C-terminal overhang region having between 9 and 50 amino acids;
L is a linker given by (Gly-Pro-Pro)₄ (SEQ ID NO: 4);
[Ac] is a chain of 0-3 amino acid residues.

17. The fibril forming peptide as recited in claim 16, wherein f_{N} consists of GPCC(Gly-Pro-Pro)₄ (SEQ ID NO: 5).

18. The fibril forming peptide as recited in claim 17, wherein f_{C} consists of GPCC and a foldon sequence.

19. The fibril forming peptide as recited in claim 18, wherein [Ac] is GTP.


SEQUENCE LISTING (5 sequences)

SEQ ID NO: 1 - protein, 108 aa, Homo sapiens
GERGPPGPQGARGLPGAPGQMGPRGLPGERGRPGAPGPAGARGEPGAPGSKGDTGAKGEPGPVGVQGPPGPAGEEGKRGARGEPGPTGPAGPKGSPGEAGRPGEAGLP

SEQ ID NO: 2 - protein, 108 aa, Homo sapiens
GPVGPAGKSGDRGETGPAGPAGPVGPVGARGPAGPQGPRGDKGETGEQGDRGIKGHRGFSGLQGPPGPPGSPGEQGPSGASGPAGPRGPPGSAGAPGKDGLNGLPGPI

SEQ ID NO: 3 - protein, 27 aa, Homo sapiens
GYIPEAPRDGQAYVRKDGEWVLLSTFL

SEQ ID NO: 4 - protein, 12 aa, Artificial Sequence
  Synthetic sequence used as a linker
GPPGPPGPPGPP

SEQ ID NO: 5 - protein, 16 aa, Artificial Sequence
  Synthetic sequence used as Fn terminal
GPCCGPPGPPGPPGPP